(19) United States
(12) Patent Application Publication
(10) Pub. No.: US 2010/0203047 A1
(43) Pub. Date: Aug. 12, 2010

(54) Novel Compositions And Methods For Stimulating Erythropoiesis In A Mammal

(75) Inventors: Koji Tamada, Lutherville, MD (US); Lieping Chen, Sparks Glencoe, MD (US)

Correspondence Address: ROYLANCE, ABRAMS, BERDO & GOODMAN, L.L.P., 1300 19TH STREET, N.W., SUITE 600, WASHINGTON,, DC, 20036, US

(73) Assignees: UNIVERSITY OF MARYLAND, BALTIMORE, Baltimore, MD (US); THE JOHNS HOPKINS UNIVERSITY, Baltimore, MD (US)

(21) Appl. No.: 12/680,819

(22) PCT Filed: Oct. 3, 2008
(86) PCT No.: PCT/US08/78769

§ 371 Date: Apr. 21, 2010

Related U.S. Application Data
(60) Provisional application No. 60/997,818, filed on Oct. 5, 2007.

(51) Int. Cl. A61K 39/395; C07K 16/18; C07K 16/28

(52) U.S. Cl. 424/133.1; 530/387.1; 530/388.22; 530/389.1; 530/387.3; 530/387.9; 424/130.1; 424/143.1

(57) ABSTRACT

The present invention relates to compositions comprising an agent which activates herpes virus entry mediator (HVEM). The present invention also relates to compositions comprising an agonist of HVEM. The present invention also relates to methods of stimulating erythropoiesis in a mammal, comprising administering to a mammal a composition comprising an agent which activates HVEM, or is an agonist of HVEM. The present invention also relates to methods for treating an anemic disorder, comprising administering to a mammal suffering therefrom an agent which activates HVEM.

### CROSS-REFERENCE TO RELATED APPLICATIONS

This application claims priority to U.S. Ser. No. 60/997,818, filed Oct. 5, 2007, which is hereby incorporated by reference in its entirety.

### STATEMENT REGARDING FEDERALLY SPONSORED RESEARCH OR DEVELOPMENT

This invention was made with government support under R01HL088954 awarded by the National Institutes of Health. The U.S. Government has certain rights in this invention.

### BACKGROUND OF THE INVENTION

1. Field of the Invention

The present invention relates to compositions and methods for stimulating erythropoietin (EPO) production in a mammal by activation of the TNF receptor superfamily protein Herpesvirus Entry Mediator (HVEM).

2. Background of the Invention

Anemia is the single-most common hematological disorder, and is caused by a deficiency of hemoglobin in erythrocytes. Because hemoglobin normally transports oxygen from the lungs to the periphery, and other organs, anemia often results in hypoxia. Under normal conditions, the amount of EPO in the circulation increases in response to hypoxia. Hypoxia may be the result of, for example, substantial blood loss through hemorrhage, destruction of red blood cells by over-exposure to radiation, reduction in oxygen intake due to high altitude or prolonged unconsciousness, or various forms of anemia. In response to such hypoxic stress, EPO levels increase, triggering erythrocyte development by stimulating the proliferation of erythroid progenitor cells. Conversely, when the number of red blood cells in the circulation is greater than that required for normal tissue oxygen consumption, EPO levels in the circulation normally decrease.

Erythropoiesis, the production of erythrocytes, occurs continuously in vivo to offset the decrease in erythrocytes by cell destruction. In wild-type animals, erythropoiesis is a highly regulated physiological mechanism that enables an adequate supply of erythrocytes to be available for proper tissue oxygenation. Naturally occurring human erythropoietin is produced in the kidney as a glycoprotein hormone with a molecular weight of about 34 kilodaltons (Id)). Naturally occurring EPO stimulates the division and differentiation of committed erythroid progenitors in the bone marrow and exerts its biological activity by binding to receptors on erythroid precursors (Krantz et al., (1991), Blood, 77:419). More specifically, during EPO-induced differentiation of erythrocyte precursor cells, globin synthesis is induced, heme complex synthesis is stimulated, and the number of ferritin receptors increases. These physiological changes allow the cell to uptake more iron and synthesize functional hemoglobin, which binds oxygen. Therefore, erythrocytes play a critical role in oxygen transfer throughout the body. Such changes are initiated by the interaction of EPO with an appropriate receptor on the cell surface of the erythrocyte precursor cells (See, e.g., Graber and Krantz (1978) Ann. Rev. Med. 29.51-66).

Because EPO is essential for red blood cell formation, EPO is useful in the treatment of blood disorders characterized by a decrease in, or a defect in, erythrocyte production. Clinically, EPO is used in the treatment of anemia. Anemia can manifest as a primary disease (e.g., sickle cell anemia), or as a secondary consequence of other disease states (e.g., chronic renal failure patients; See Eschbach et al., (1987), NEJM, 316:73-78; Egrie et al., (1988), Kidney Intl., 33:262; Lim et al., (1989), Ann. Intern. Med., 110:108-114, all incorporated herein by reference in their entirety). Further, anemia may manifest as a side effect of a pre-existing therapeutic regimen. For example, patients with AIDS and malignancies often develop anemia as a consequence of therapeutic intervention (Danna et al., In: M B, Garnick, ed. Erythropoietin in Clinical Applications-An International Perspective. New York, N.Y.: Marcel Dekker; 1990: p. 301-324).

Recent studies have also provided a basis for the administration of therapy to stimulate EPO production in a variety of disease states, disorders, and states of hematologic irregularity, including: beta-thalassemia (see, Vedovato et al., (1984), Acta. Haematol., 71:211-213); cystic fibrosis (see, Vichinsky et al., (1984), J. Pediatric., 105:15-21); pregnancy and menstrual disorders (see, Cotes et al., (1983), Brit. J. Ostet. Gyneacol., 90:304-311); spinal cord injury (see, Claus-Walker et al., (1984), Arch. Phys. Med. Rehabil., 65:370-374); acute blood loss (see, Miller et al., (1982), Brit. J. Haematol., 52:545-590); neoplastic disease states accompanied by abnormal erythropoiesis (see, Dainiak et al., 1983, Cancer, 5:1101-1106 and Schwartz et al., (1983), Otolaryngol., 109:269-272); and renal insufficiency (see, Eschbach et al., (1987), N. Eng. J. Med., 316:73-78).

As a therapeutic compound, EPO has been manufactured biosynthetically using recombinant DNA technology (Egrie et al., (1986), Immunobiol., 72:213 224), and is the product of a cloned human EPO gene expressed in chinese hamster ovary cells (CHO cells). The biological effect of EPO is mediated, at least in part, through interaction with the EPO receptor (EPO-R). The genes encoding the murine and human EPO receptor are known, and are described in PCT Pub. No. WO 90/08822. It has been suggested that binding of EPO to EPO-R, results in the dimerization and activation of two EPO-R molecules, which results in subsequent steps of signal transduction (see, Watowich et al., (1992), Proc. Natl. Acad. Sci. USA, 89:2140-2144).

Further, alternative EPO-R binding compounds for the purpose of increasing EPO production have been identified and described, for example in U.S. Pat. Nos. 5,773,569; 5,830,851; and 5,986,047; PCT Pub. No. WO 96/40749; U.S. Pat. No. 5,767,078 and PCT Pub. No. 96/40772; PCT Pub. No. WO 01/38342; and WO 01/91780, all incorporated herein by reference in their entireties.

Given the critical role of EPO in erythrocyte development, there remains a need for efficacious therapies able to stimulate erythropoiesis in vivo. The methods and compositions of the present invention provide such therapies by activating Herpesvirus Entry Mediator (HVEM), which results in stimulation of erythropoiesis.

HVEM is a member of the tumor necrosis factor-receptor (TNF-R) superfamily, and was originally identified as a cellular receptor required for entry of Herpes Simplex Virus (HSV), by binding to HSV glycoprotein D. HVEM is widely expressed on the surface of immune cells and has also been identified on certain non-hematopoietic cells. HVEM was identified by expression cloning of several HeLa cell products which, when expressed in otherwise nonpermissive CHO cells, rendered the CHO cells susceptible to entry by many HSV strains. Several studies suggest that HVEM is involved in activation of the host immune response. For example, HVEM is predominantly expressed in lymphocyte-rich tissues, and binding of HVEM to several members of the TNFR-associated factor (TRAF) family of proteins activates transcriptional regulators such as nuclear factor KB (NF-κB), Jun N-terminal kinase, and AP-1. Moreover, HVEM binds to lymphotoxin-alpha (also known as tumor necrosis factor beta) and to a membrane-associated protein designated LIGHT (Lymphotoxin-like, exhibits inducible expression, and competes with HSV glycoprotein D for HVEM, a receptor expression by T lymphocytes). The structure of HVEM has been identified for several mammalian species, such as human (NCBI Accession No. AAQ89238; SEQ ID NO: 3), mouse (NCBI Accession No. AAQ08183), rhesus monkey (NCBI Accession No. ABI13587) and Norway rat (NCBI Accession No. NP_{—}001015034), and has been predicted based on nucleotide sequence for several other mammalian species.

LIGHT is a lymphotoxin homolog, and is expressed by T cells upon induction with phorbol 12-myristate 13-acetate (PMA) (see, Mauri et al. (1998), Immunity 8:21-30; Marsters et al. (1997), J. Biol. Chem. 272:14029-14032); Hsu et al. (1997), J. Biol. Chem. 272:13471-13474). HVEM is believed to trimerize upon binding to its trimeric ligands (e.g., LIGHT), which binding is mediated by cysteine-rich domains (CRDs) of HVEM. LTα binds to HVEM through interactions with CRD2 and CRD3. Several antibody-blocking and peptide-mapping studies have demonstrated that LIGHT and LTα binding to HVEM can be mediated through binding to CRD2 and CRD3. Ligation of HVEM (by LIGHT, for example) leads to the recruitment of TRAFs 1, 2, 3, and 5, subsequently up-regulating the expression of NF-κB and AP-1 transcription factors that mediate proliferation, survival, and cytokine production.

At present, most studies on HVEM have been directed to understanding the role of HVEM in regulating T-lymphocyte activation in response to invading pathogens in the immune system. Little is known of the role of HVEM, and its downstream signaling pathways, in other cell types, such as bone marrow cells. The present invention provides novel compositions and methods for stimulating erythropoietin (EPO) production in a mammal, by activation of HVEM.

All publications, patent applications, patents, and other references mentioned herein are incorporated by reference in their entirety.

### SUMMARY OF THE INVENTION

The present invention relates to compositions for stimulating erythropoiesis in a mammal comprising an agent which activates herpes virus entry mediator (HVEM).

The present invention also relates to compositions for stimulating erythropoiesis in a mammal comprising an agonist of HVEM.

The present invention further relates to compositions for stimulating erythropoiesis in a mammal comprising an agonist of HVEM, wherein the agonist may be an antibody, a fusion protein of a cognate ligand, such as LIGHT or Lymphotoxin alpha, DNA Aptamers, RNA Aptamers, or a small molecule.

The present invention relates to methods of stimulating erythropoiesis in a mammal, comprising administering to a mammal a composition comprising an agent which activates HVEM.

The present invention also relates to methods of stimulating erythropoiesis in a mammal, comprising administering to a mammal a composition comprising an agent which is an agonist of HVEM.

The present invention further relates to methods of stimulating erythropoiesis in a mammal, comprising administering to a mammal a composition comprising an agonist of HVEM, wherein the agonist may be an antibody, a fusion protein of a cognate ligand, such as LIGHT or Lymphotoxin alpha, DNA Aptamers, RNA Aptamers, or a small molecule.

The present invention relates to methods and compositions useful for treating an anemic disorder comprising administering to a mammal suffering therefrom an agent which activates HVEM. In particular embodiments, an anemic disorder is a result of, for example, renal failure, chemotherapy or drug treatment.

The present invention also relates to methods and compostions useful for treating a neurodegerative disorder including Parkinson's disease and Huntington's disease, comprising administering to a mammal suffering therefrom an agent which activates HVEM.

The present invention also further relates to methods and compositions to treat an inflammatory disorder comprising administering to a mammal suffering therefrom an agent which activates HVEM, wherein the inflammatory disorder is selected from the group consisting of asthma, allergy, rheumatoid arthritis and inflammatory bowel disorder.

The present invention relates to an antibody that specifically binds to HVEM and stimulates erythropoiesis in a mammal. The antibody is an agonist of a mammalian HVEM and is an antibody selected from the group consisting of a monoclonal antibody, a polyclonal antibody, a humanized antibody, and an antigen-binding antibody fragment.

The present invention also relates to an antibody that specifically binds to a human HVEM and stimulates erythropoiesis in a mammal. The antibody specifically binds to a polypeptide comprising the amino acid sequence of SEQ ID NO: 3. In further aspects, the antibody stimulates erythropoiesis.

The present invention also further relates to an antibody that specifically binds to a mammalian HVEM, wherein said antibody is an agonist of said mammalian HVEM, produced by a method comprising (i) immunizing a mammal with a mammalian HVEM protein, or the extracellular domain thereof; (ii) isolating an antibody from said mammal that binds said mammalian HVEM protein, or extracellular domain thereof; (iii) contacting the antibody of step (ii) with a T-lymphocyte in the presence of anti-CD3 antibody; (iv) determining whether step (iii) induces proliferation of said T-lymphocyte; wherein when the proliferation of said T-lymphocyte is induced, the antibody isolated in step (ii) is an agonist of HVEM.

### BRIEF DESCRIPTION OF THE DRAWINGS

FIG. 1 depicts HVEM-agonist induced expansion of Ter-119⁺ erythroid lineage cells. (A) FACS analysis of splenocytes stained with anti-Ter-119 antibody and anti-CD45 antibody. (B) Expansion of Ter-119⁺/CD45⁻ erythroid lineage cells in wild-type mice administered HM3.30 IgG antibody. HVEM knockout mice (HVEM^{-/-}) exhibited no expansion of Ter-119⁺/CD45⁻ erythroid lineage cells when administered HM3.30 IgG antibody. (C) Expansion of Ter-119⁺/CD45⁻ erythroid lineage cells in wild-type mice requires an antibody agonistic to HVEM. A non-agonistic antibody (HM2.2) produced considerably weaker expansion of Ter-119⁺/CD45⁻ erythroid lineage cells than agonistic antibody HM3.30. (D) Expansion of Ter-119⁺/CD45⁻ erythroid lineage cells in wild-type mice, LIGHT^{-/-} mice and LTα^{-/-} mice administered HM3.30 IgG antibody.

FIG. 2 depicts that the expansion of Ter-119⁺/CD45⁻ erythroid lineage cells originates in the bone marrow. (A) Analysis of Ter-119⁺/CD45⁻ erythroid lineage cells in bone marrow, spleen, and peripheral blood 2, 4, and 6 days after administration of HM3.30 IgG antibody (closed circles) or control IgG antibody (open circles). (B) Analysis of benzidine positive, CFU-E numbers in HM3.30 IgG and control hamster IgG injected mice. (C) Analysis of Ter-119⁺/CD45⁻ erythroid lineage cells in the bone marrow and blood of splenectomized mice.

FIG. 3 depicts FACS analysis of expression of HVEM on bone marrow cells using anti-Ter-119 antibody.

FIG. 4 depicts EPO production in wild-type mice administered HM3.30 IgG antibody. (A) Measurement of the concentration of EPO in the sera of wild-type mice after administration of HM3.30 IgG antibody. (B) Inhibition of expansion of Ter-119⁺/CD45⁻ erythroid lineage cells by HM3.30 IgG antibody in the presence of anti-EPO antibody.

FIG. 5 depicts the requirement for HVEM in HM3.30 induced erythropoiesis in bone marrow derived cells. Chimeric mice containing either wild-type or HVEM^{-/-} bone marrow cells were administered either HM3.30 IgG antibody, or control IgG antibody, and expansion of Ter-119⁺/CD45⁻ erythroid lineage cells (A) or EPO production (B) was measured. The expansion of Ter-119⁺/CD45⁻ erythroid lineage cells and production of EPO could be stimulated if the HVEM^{-/-} mice were reconstituted with wild-type bone marrow cells (C and D).

FIG. 6 depicts HM3.30 induced erythropoiesis in the absence of lymphocytes or neutrophils. (A) Analysis of the expansion of Ter-119⁺/CD45⁻ erythroid lineage cells in Rag-deficient mice administered HM3.30 IgG antibody or control IgG antibody. (B) Analysis of the expansion of Ter-119⁺/CD45⁻ erythroid lineage cells in Rag-deficient mice, also depleted for neutrophils, administered HM3.30 IgG antibody or control IgG antibody

FIG. 7 depicts inhibition of expansion of Ter-119⁺/CD45⁻ erythroid lineage cells by the eNOS inhibitor, L-NAME. Wild-type mice were administered L-NAME, and the expansion of Ter-119⁺/CD45⁻ erythroid lineage cells was measured following administration of HM3.30 antibody or control IgG antibody.

FIG. 8 depicts the increase in EPO and hemoglobin when anemic mice are treated with HM3.30 IgG antibody. Wild-type mice were made anemic by administration of Cisplatin, then treated with HM3.30 antibody. Serum levels of EPO (A) and hemoglobin (B) were measured. (C) HVEM^{-/-} and wild-type mice were treated with 5-fluorouracil, and the level of EPO produced was measured. HVEM^{-/-} mice produce less EPO than wild-type mice when administered 5-fluorouracil.

FIG. 9 depicts the binding specificity of HM3.30 IgG antibody. 293T cells transfected with plasmids encoding mouse HVEM, human HVEM, a chimeric gene of cysteine-rich domain (CRD) 1 of human HVEM fused to CRDs 2-4 of mouse HVEM, or a chimeric gene of CRD 1 of mouse HVEM fused to CRDs 2-4 of human HVEM. The transfected cells were labeled with HM3.30 IgG antibody, followed by PE-conjugated anti-hamster IgG, and binding was determined by FACS analysis.

FIG. 10 depicts the characterization of two isolated agonistic anti-human HVEM antibodies. (A) 293T cells expressing human HVEM were stained with culture supernatants of hybridoma clones 27 and 6H9, followed by staining with PE-conjugated anti-mouse IgG Ab. Binding was determined by FACS analysis. (B) Agonistic activity of antibody clones 27 and 6H9 was determined by co-incubation of human T-cells with anti-CD3 antibody and antibody clones 27 and 6H9. T-cell proliferation was determined 3 days after administration of antibody by measuring 3H-thymidine incorporation.

### DETAILED DESCRIPTION OF THE INVENTION

Unless otherwise noted, the terms used herein are to be understood according to conventional usage by those of ordinary skill in the relevant art. In addition to the definitions of terms provided below, definitions of common terms in molecular biology may also be found in: Rieger et al., (1991) Glossary of genetics: classical and molecular, 5th Ed., Berlin: Springer-Verlag; and in Current Protocols in Molecular Biology; F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “an antibody” can mean that at least one antibody can be utilized.

As used herein, the term “activates Herpes virus entry mediator (HVEM)” refers to the induction of downstream signaling pathways by activation of HVEM. Such downstream signaling pathways may include, but are not limited to, transcriptional regulators such as nuclear factor KB (NF-κB), Jun N-terminal kinase, and AP-1. In particular embodiments, an agent (including, for example, an antibody of the invention) that activates HVEM has at least one other function including the stimulation of erythropoiesis.

As used herein, the term “agonist” refers to peptide and non-peptide ligands that bind to HVEM, and which have the ability to induce HVEM to activate downstream signaling pathways. Such downstream signaling pathways may include, but are not limited to, transcriptional regulators such as nuclear factor KB (NF-κB), Jun N-terminal kinase, and AP-1. Preferably, the agonist is a peptide agonist. More preferably, the agonist is a peptide agonist selected from the group consisting of LIGHT (Lymphotoxin-like, exhibits inducible expression, and competes with HSV glycoprotein D for HVEM, a receptor expression by T lymphocytes), Lymphotoxin alpha (LTα), and an agonistic antibody.

As used herein, the term “agonistic antibody” refers to an antibody that binds to HVEM, and which has the ability to induce HVEM to activate downstream signaling pathways. Such downstream signaling pathways may include, but are not limited to, transcriptional regulators such as nuclear factor KB (NF-κB), Jun N-terminal kinase, and AP-1. For example, the agonistic antibody may bind to the extracellular domain of HVEM, preferably at least to one CRD. The term “agonistic antibody” encompasses anti-HVEM agonistic monoclonal antibodies and anti-HVEM agonistic antibody compositions with polyepitopic specificity. In a preferred embodiment of the invention, the antibodies are monoclonal antibodies. The antibodies may be raised against any mammalian HVEM. Preferably, the antibody is raised against a primate, rodent, canine, feline, equine, bovine or porcine HVEM. More preferably, the antibody is raised against a human HVEM. In certain embodiments, an antibody of the invention causes stimulation of erythropoiesis. In certain embodiments, the antibody that stimulates erythropoiesis is an agonist of HVEM.

As used herein, the term “antibodies” refers to proteins which exhibit binding specificity to a specific antigen. Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy chain has at one end a variable domain followed by a number of constant domains. Each light chain has a variable domain at one end and a constant domain at its other end; the constant domain of the light chain is aligned with the first constant domain of the heavy chain, and the light chain variable domain is aligned with the variable domain of the heavy chain.

As used herein, the term “antibody” is used in the broadest sense and encompasses single monoclonal antibodies, antibody compositions with polyepitopic specificity (i.e, polyclonal antibody compositions), chimeric antibodies, humanized antibodies, single-chained antibodies, and antibody fragments (e.g., Fab, F(ab′), Fv), so long as the antibodies exhibit the desired agonistic activity and stimulate erythropoiesis.

The term “variable” refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies (see Kabat, E. A. et al., (1987), Sequences of Proteins of Immunological Interest National Institute of Health, Bethesda, Md.). The constant domains are not involved directly in binding an antibody to an antigen, but exhibit various effector functions, such as participation of the antibody in antibody-dependent cellular toxicity.

Papain digestion of antibodies produces two identical antigen binding fragments, called Fab fragments, each with a single antigen binding site, and a residual “Fc” fragment. Pepsin treatment yields an F(ab′) fragment that has two antigen combining sites and is still capable of cross-linking antigen.

As used herein, the term “Fv” refers to the minimum antibody fragment which contains a complete antigen recognition and binding site. This region consists of a dimer of one heavy and one light chain variable domain in a non-covalent association. However, it is recognized in the art that even a single variable domain (or half of an Fv comprising only three CDRs specific for an antigen) has the ability to recognize and bind antigen.

As used herein, the term “Fab fragment” refers to an antigen-binding fragment containing the constant domain of the light chain and the first constant domain of the heavy chain. Fab′ fragments differ from Fab fragments by the addition of a few residues at the carboxy terminus of the heavy chain constant domain including one or more cysteines from the antibody hinge region. As used herein, the term “Fab′-SH” refers to Fab′ fragments in which the cysteine residue(s) of the constant domains bear a free thiol group.

The light chains of antibodies from any vertebrate species can be assigned to one of two clearly distinct types, called kappa and lambda, based on the amino acid sequences of their constant domains.

Depending on the amino acid sequence of the constant domain of their heavy chains, antibodies (immunoglobulins) can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. In contrast to polyepitopic (polyclonal) antibody preparations, which typically include different antibodies directed against different epitopes, each monoclonal antibody is directed against a single epitope of the antigen. Monoclonal antibodies can be produced and isolated from a hybridoma culture. The term “monoclonal” indicates the specificity of the antibody, and is not to be construed as requiring production of the antibody by any particular method. For example, the monoclonal antibodies to be used in accordance with the present invention may be made by the hybridoma method first described by Kohler et al., (1975), Nature, 256:495, or may be made by recombinant DNA methods (see, e.g., U.S. Pat. No. 4,816,567).

The monoclonal antibodies contemplated herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired agonistic activity (see, e.g. U.S. Pat. No. 4,816,567 and Morrison et al., (1984), Proc. Natl. Acad. Sci. USA, 81:6851-6855).

As used herein, the term “humanized antibody” refers to forms of non-human (e.g., murine) antibodies, chimeric antibodies, immunoglobulin chains or fragments thereof (such as Fv, Fab, Fab′, or other antigen-binding subsequences of antibodies) which contain minimal sequence derived from non-human immunoglobulin. For example, humanized antibodies may be human immunoglobulins in which amino acid residues from a complementarity determining region (CDR) are replaced by residues from a CDR of a non-human species (i.e., a donor antibody) such as a mouse, rat, goat, donkey or rabbit having the desired specificity and affinity. In some embodiments, Fv framework residues of the human immunoglobulin are replaced by corresponding non-human residues. Furthermore, humanized antibodies may comprise residues which are found neither in the recipient antibody, nor in the imported CDR or framework sequences. Such modifications may be introduced to further refine and optimize antibody performance, and are well-known in the art. Preferably, the humanized antibody will comprise at least one, and more preferably two, variable domains, in which all or substantially all of the CDR regions correspond to those of a non-human immunoglobulin and all or substantially all of the FR regions are those of a human immunoglobulin consensus sequence. Even more preferably, the humanized antibody will also comprise at least a portion of a human immunoglobulin constant region (Fc). For further details see: US Patent Application Publication No. 20030096403; US Patent Application Publication No. 20030144483; US Patent Application Publication No. 20080027147; U.S. Pat. No. 5,225,539; Jones et al., (1986), Nature, 321:522-525; Reichmann et al., (1988), Nature, 332:323-329; and Presta et al. (1992), Curr. Op. Struct. Biol., 2:593-596).

In one embodiment, the antibodies of the claimed invention are monoclonal, and stimulate erythropoiesis. In another embodiment, the antibodies of the claimed invention bind to human HVEM in an agonistic manner to stimulate erythropoiesis. In another embodiment, the antibody exhibits the same biological characteristics as any one of monoclonal antibodies HM3.30, clone 27 and clone 6H9, set forth herein. The term “biological characteristics” is intended to refer to the in vitro and/or in vivo activities of the monoclonal antibody, e.g., the ability to bind to human HVEM in an agonistic manner, the ability to activate downstream signaling pathways, which may include, but are not limited to, transcriptional regulators such as nuclear factor KB (NF-κB), Jun N-terminal kinase, and AP-1, and the ability to stimulate EPO production. In another embodiment, the antibody of the claimed invention binds to the same epitope as any one of monoclonal antibodies HM3.30, clone 27 and clone 6H9, set forth herein. To determine whether a monoclonal antibody has the same binding specificity as one of monoclonal antibodies HM3.30, clone 27 and clone 6H9, one can, for example, use a competitive ELISA binding assay.

As used herein the term “isolated” is intended to refer to an agent, such as a polypeptide or an antibody which has been identified and separated and/or recovered from a component of its natural environment. For example, an “isolated” monoclonal antibody of the present invention may be purified from a cell culture or other synthetic environment, preferably to a purity greater than 95% by weight of protein as determined by the Lowry method, or to homogeneity by SDS-PAGE under reducing or nonreducing conditions using Coomassie blue or silver stain.

As used herein, the term “effective amount” is used to refer to an amount of any given molecule sufficient for the prevention, treatment, or desired outcome of a specified physiological condition or symptom. The effective amount of the agent or agonist to be administered will be governed by considerations such as the disorder being treated, the particular mammal being treated, the clinical condition of the individual patient, the cause of the disorder, the site of delivery of the agent, the method of administration, the scheduling of administration, and other factors well-known to medical practitioners. In one embodiment, the effective amount is the minimum amount necessary to stimulate erythropoiesis in a mammal. Preferably, the mammal is a primate, rodent, canine, feline, equine, bovine and porcine. More preferably, the mammal is a human.

In one embodiment, the effective amount is the minimum amount necessary to treat an anemic disorder in a mammal. Preferably, the mammal is a primate, rodent, canine, feline, equine, bovine and porcine. More preferably, the mammal is a human. In certain embodiments, the anemic disorder is the result of, for example, renal failure, chemotherapy, drug treatment, blood loss, diet, malnutrition, pregnancy, HIV/AIDS associated anemia, infection, and a genetic or acquired anemic disorder including, for example, sickle cell anemia, thalassemias, hemolytic anemia, and aplastic anemia. Other diseases and conditions encompassed by the invention are described in US Patent Application Publication No. 20060178317.

In another embodiment, the effective amount is the minimum amount necessary to treat a neurodegenerative disorder in a mammal. Preferably, the mammal is a primate, rodent, canine, feline, equine, bovine and porcine. More preferably, the mammal is a human. In certain embodiments, the neurodegenerative disorder is selected from the group consisting of Parkinson's disease, Huntington's disease, Alzheimer's, schizophrenia, and amyotrophic lateral sclerosis.

In yet another embodiment, the effective amount is the minimum amount necessary to treat acute ischemic or hemorrhagic stroke in a mammal. Preferably, the mammal is a primate, rodent, canine, feline, equine, bovine and porcine. More preferably, the mammal is a human.

In another embodiment, the effective amount is the minimum amount necessary to treat an inflammatory disorder in a mammal. Preferably, the mammal is a primate, rodent, canine, feline, equine, bovine and porcine. More preferably, the mammal is a human. In certain embodiments, the inflammatory disorder is selected from the group consisting of asthma, allergy, rheumatoid arthritis and inflammatory bowel disorder.

As used herein, the term “aptamer” refers to an oligonucleotide that is capable of forming a complex with a target substance. Preferably, the aptamer forms a complex with HVEM and is an agonist of HVEM. The aptamer may be prepared by any known method, including synthetic, recombinant, and purification methods, and may be used alone or in combination with other aptamers specific for the same target.

The aptamers of the present invention may interact or complex with HVEM through hydrogen bonding or other molecular forces. However, such interacting or complexing excludes the normal “Watson-Crick”-type binding interactions (i.e., adenine-thymine and guanine-cytosine base-pairing) traditionally associated with DNA duplex formation. In general, a minimum of approximately 10 nucleotides is necessary to effect specific binding. Although the oligonucleotides generally described herein are single-stranded or double-stranded, it is contemplated that aptamers may sometimes assume triple-stranded or quadruple-stranded structures.

The aptamers of the present invention may be synthesized as conventional DNA or RNA moieties, or may be “modified” oligomers which are recognized in the art. Such modifications include, but are not limited to, the incorporation of modified or analogous forms of sugars (ribose and deoxyribose), alternative linking groups, or analogous forms of purine and pyrimidine bases.

As used herein, the term “fusion protein” refers to the product of a gene in which the coding sequence for one protein is extensively altered by fusing at least a part of it to the coding sequence of a second protein from a different gene. The fusion partner may serve various functions, including, but not limited to enhancement of the solubility of the protein that activates HVEM, or is an agonist of HVEM, as well as providing an “affinity tag” to allow purification of the recombinant fusion protein from the host cell or culture supernatant, or both.

As used herein, the term “Ter-119⁺ cells” refers to cells which express the epitope specifically bound by the Ter-119 monoclonal antibody as described in Kina et al., (2000), Br. J. Haematol., 109:280-7). In wild-type adult mice, Ter-119 antibody reacts with mature erythrocytes, 20-25% of bone marrow cells and 2-3% of spleen cells, but not with thymocytes nor lymph node cells.

As used herein, the term “administering” refers to any process of introducing a composition into or onto the body of a mammal and includes, but is not limited to, particular methods described in this specification.

As used herein, the term “erythropoietin (EPO) receptor agonist” refers to molecules capable of causing activation of the EPO receptor, which may result from any number of mechanisms. EPO receptor agonists include, but are not limited to, darbepoietin alfa (ARANESP™), epoietin alfa (EPO, EPOGEN™), and anti-EPO receptor agonist antibodies (see, for example, U.S. Pat. No. 7,087,224; WO/2000/024893).

As used herein, a “functional fragment” is a fragment or splice variant of a full length polypeptide that exerts a similar physiological or cellular effect as the full length polypeptide. The biological effect of the functional fragment need not be identical in scope or strength as the full-length polypeptide, so long as a similar physiological or cellular effect is seen. For example, a functional fragment of an anti-HVEM antibody can detectably activate HVEM.

As used herein, the term “variant” includes chimeric or fusion polypeptides, homologs, analogs, orthologs, and paralogs. In addition, a variant of a reference protein or polypeptide is a protein or polypeptide whose amino acid sequence is at least about 80% identical to the reference protein or polypeptide. In specific embodiments, the variant is at least about 85%, 90%, 95%, 95%, 97%, 98%, 99% or even 100% identical to the reference protein or polypeptide. As used herein, the terms “correspond(s) to” and “corresponding to,” as they relate to sequence alignment, are intended to mean enumerated positions within the reference protein or polypeptide, e.g., wild-type human or mouse HVEM, and those positions in the modified protein or polypeptide that align with the positions on the reference protein or polypeptide. Thus, when the amino acid sequence of a subject protein or polypeptide is aligned with the amino acid sequence of a reference protein or polypeptide, the sequence that “corresponds to” certain enumerated positions of the reference protein or polypeptide sequence are those that align with these positions of the reference sequence, but are not necessarily in these exact numerical positions of the reference sequence. Methods for aligning sequences for determining corresponding amino acids between sequences are described below.

A polypeptide having an amino acid sequence at least, for example, about 95% “identical” to a reference an amino acid sequence encoding, for example a mammalian HVEM, is understood to mean that the amino acid sequence of the polypeptide is identical to the reference sequence except that the amino acid sequence may include up to about five modifications per each 100 amino acids of the reference amino acid sequence encoding the reference HVEM. In other words, to obtain a peptide having an amino acid sequence at least about 95% identical to a reference amino acid sequence, up to about 5% of the amino acid residues of the reference sequence may be deleted or substituted with another amino acid or a number of amino acids up to about 5% of the total amino acids in the reference sequence may be inserted into the reference sequence. These modifications of the reference sequence may occur at the N-terminus or C-terminus positions of the reference amino acid sequence or anywhere between those terminal positions, interspersed either individually among amino acids in the reference sequence or in one or more contiguous groups within the reference sequence.

As used herein, “identity” is a measure of the identity of nucleotide sequences or amino acid sequences compared to a reference nucleotide or amino acid sequence. In general, the sequences are aligned so that the highest order match is obtained. “Identity” per se has an art-recognized meaning and can be calculated using published techniques. (See, e.g., Computational Molecular Biology, Lesk, A. M., ed., Oxford University Press, New York (1988); Biocomputing: Informatics And Genome Projects, Smith, D. W., ed., Academic Press, New York (1993); Computer Analysis of Sequence Data, Part I, Griffin, A. M., and Griffin, H. G., eds., Humana Press, New Jersey (1994); von Heinje, G., Sequence Analysis In Molecular Biology, Academic Press (1987); and Sequence Analysis Primer, Gribskov, M. and Devereux, J., eds., M Stockton Press, New York (1991)). While there exist several methods to measure identity between two polynucleotide or polypeptide sequences, the term “identity” is well known to skilled artisans (Carillo, H. & Lipton, D., Siam J Applied Math 48:1073 (1988)). Methods commonly employed to determine identity or similarity between two sequences include, but are not limited to, those disclosed in Guide to Huge Computers, Martin J. Bishop, ed., Academic Press, San Diego (1994) and Carillo, H. & Lipton, D., Siam J Applied Math 48:1073 (1988). Computer programs may also contain methods and algorithms that calculate identity and similarity. Examples of computer program methods to determine identity and similarity between two sequences include, but are not limited to, GCG program package (Devereux, J., et al., (1984), Nucleic Acids Research 12(i):387), BLASTP, ExPASy, BLASTN, FASTA (Atschul, S. F., et al., (1990), J Molec Biol., 215:403) and FASTDB. Examples of methods to determine identity and similarity are discussed in Michaels, G. and Garian, R., Current Protocols in Protein Science, Vol 1, John Wiley & Sons, Inc. (2000), which is incorporated by reference. In one embodiment of the present invention, the algorithm used to determine identity between two or more polypeptides is BLASTP.

I. Production of Antibodies

Polyclonal antibodies to HVEM may be raised in animals using any of the techniques well-known in the art for antibody production. For example, polyclonal antibodies may be generated by multiple subcutaneous (sc) or intraperitoneal (ip) injections of HVEM, or a fragment thereof, and an adjuvant. Under certain conditions, it may be useful to conjugate the HVEM, or fragment thereof, to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), and glutaraldehyde. Aggregating agents such as alum may also be used to enhance the immune response.

Monoclonal antibodies of the present invention may be made using the hybridoma method first described by Kohler et al., (1975), Nature, 256:495), or may be made by recombinant DNA methods (see e.g., U.S. Pat. No. 4,816,567).

In the hybridoma method, a mouse or other appropriate host animal, such as hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma [Goding, Monoclonal Antibodies: Principles and Practice, pp. 59-103 (Academic Press, 1986)].

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. U.S.A., and SP-2 cells available from the American Type Culture Collection, Rockville, Md. U.S.A. Human myeloma and mouse-human heteromyeloma cell lines also have been described for the production of human monoclonal antibodies (Kozbor et al., (1984), J. Immunol., 133:3001; Brodeur et al., (1987), Monoclonal Antibody Production Techniques and Applications, pp. 51-63, Marcel Dekker, Inc., New York.

Culture medium in which hybridoma cells are growing can be assayed for production of monoclonal antibodies that bind the immunogen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). After hybridoma cells are identified that produce antibodies of the desired specificity, the clones may be subcloned by limiting dilution procedures and grown by standard methods. Suitable culture media for this purpose include, for example, Dulbecco's Modified Eagle's Medium or RPMI-1640 medium. In addition, the hybridoma cells may be grown in vivo as ascites tumors in an animal.

Preferably, the monoclonal antibodies secreted by the subclones are separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, (see Morrison et al., (1984), Proc. Nat. Acad. Sci., 81:6851), or by covalently joining to the immunoglobulin coding sequence all or part of the coding sequence for a non-immunoglobulin polypeptide.

Methods for humanizing non-human antibodies are well known in the art. Generally, a humanized antibody has one or more amino acid residues introduced into it from a non-human antibody. Humanization can be essentially performed following the method of Winter and co-workers (Jones et al., (1986), Nature, 321:522-525; Riechmann et al., (1988), Nature, 332:323-327; Verhoeyen et al., (1988), Science, 239:1534-1536), by substituting rodent CDRs or CDR sequences for the corresponding sequences of a human antibody.

The choice of human variable domains, both light and heavy, to be used in making the humanized antibodies is an important consideration in reducing antibody antigenicity. In the “bestfit” method, the sequence of the variable domain of a non-human antibody is screened against the entire library of known human variable domain sequences. The human sequence which is closest to that of the non-human sequence is then accepted as the human framework (FR) for the humanized antibody (Sims et al., (1993), J. Immunol., 151:2296; Chothia et al., (1987), J. Mol. Biol., 196:901). In another method, a particular framework derived from the consensus sequence of all human antibodies of a particular subgroup of light or heavy chains may be used. The same framework may be used for several different humanized antibodies (Carter et al., (1992), Proc. Natl. Acad. Sci. USA, 89:4285; Presta et al., (1993), J. Immunol. 151:2623).

II. Screening for Activators of HVEM

To screen for agents that activate HVEM, such as agonistic antibodies, the agent may be initially tested for specific binding of the agent to HVEM using any of the well-known binding assays known in the art. Such assays include using plasmon resonance to measure the binding of an agent to HVEM (i.e., using a BIAcore Instrument), or radiolabeling or fluorescence-labeling of the agent to directly assess binding. For the detection of antibodies that specifically bind HVEM, assays such as enzyme-linked immunosorbent assay (ELISA), radioimmunoassay (RIA) and western blotting may be employed. To determine whether an agent that binds HVEM is an agonist of HVEM, T-lymphocytes expressing HVEM can be incubated in vitro in the combined presence of anti-CD3 antibody and the agent to be tested for HVEM activation. In such a method, the induction of proliferation of the T-lymphocytes indicates that the agent is an agonist of HVEM. T-lymphocyte proliferation can be measured using any of the assays well-known in the art for such a purpose, such as, for example, measurement of the incorporation of radiolabeled 3H-thymidine, flow cytometric assessment of CD38 expression, and by an enzyme-linked immunosorbent assay (ELISA) based on bromo-2′-deoxyuridine (BrdU) incorporation.

In other methods for determining whether an agent can activate HVEM, cells, such as T-lymphocytes, may be obtained from HVEM^{+/+} and HVEM^{-/-} animals, and exposed to the agent. Activation of HVEM can be measured either through use of the proliferation assay using anti-CD3 antibody, discussed above, or by measuring downstream signaling events known in the art to result from HVEM activation, such as the activation of transcriptional regulators such as nuclear factor KB (NF-κB), Jun N-terminal kinase, and AP-1. The specificity of the activation of HVEM can be determined by comparing the results to the HVEM^{-/-} cells treated with the agent.

III. Stimulating Erythropoiesis by HVEM Activation

The present invention also relates to methods of stimulating erythropoiesis in a mammal. In one embodiment, an agent which activates HVEM, or which is an agonist of HVEM, may be administered to a mammal to increase erythropoiesis. An increase in erythropoiesis can be determined by measuring the concentration of EPO in the serum of the mammal before and after treatment with the agent which activates HVEM, or which is an agonist of HVEM, or by measuring the hemoglobin or erythrocyte level in the blood.

In other embodiments, the stimulation of erythropoiesis by administering an agent which activates HVEM, more preferably an agonist of HVEM, may be used for the treatment of blood disorders characterized by a decrease in, or a defect in, erythrocyte production. For example, EPO is an art-recognized treatment for anemia. Anemia may manifest as a primary disease (e.g., sickle cell anemia), or as a secondary consequence of other disease states (e.g., chronic renal failure patients; See Eschbach et al. (1987), NEJM, 316:73-78; Egrie et al. (1988), Kidney Intl., 33:262; Lim et al. (1989), Ann. Intern. Med. 110:108-114, all incorporated herein by reference in their entirety). Further, anemia may manifest as a side effect of a pre-existing therapeutic regimen. For example, patients with AIDS and malignancies often develop anemia as a consequence of therapeutic intervention (Danna et al., In: M B, Garnick, ed. Erythropoietin in Clinical Applications—An International Perspective. New York, N.Y.: Marcel Dekker; 1990: p. 301-324). The present invention thus provides a method for the treatment of anemia by administering an agent which activates HVEM, preferably an agonist of HVEM. Preferably, the mammal is a primate, rodent, canine, feline, equine, bovine or porcine. More preferably, the mammal is a human. In certain embodiments, the anemia results from renal failure, chemotherapy or drug treatment.

In another embodiment of the present invention, an agent which activates HVEM, preferably an agonist of HVEM, is administered to treat a neurodegenerative disorder in a mammal. Preferably, the mammal is a primate, rodent, canine, feline, equine, bovine or porcine. More preferably, the mammal is a human. In certain embodiments, the neurodegenerative disorder is selected from the group consisting of Parkinson's disease and Huntington's disease.

In yet another embodiment of the present invention, an agent which activates HVEM, preferably an agonist of HVEM, is administered to treat acute ischemic or hemorrhagic stroke in a mammal. Preferably, the mammal is a primate, rodent, canine, feline, equine, bovine or porcine. More preferably, the mammal is a human.

In some embodiments of the present invention, an agent which activates HVEM, preferably an agonist of HVEM, is administered to treat inflammatory disorders in a mammal. Preferably, the mammal is a primate, rodent, canine, feline, equine, bovine or porcine. More preferably, the mammal is a human. In certain embodiments, the inflammatory disorder is selected from the group consisting of asthma, allergy, rheumatoid arthritis and inflammatory bowel disorder.

In certain embodiments, the invention provides for pharmaceutical compositions comprising a pharmaceutically acceptable diluent, carrier, solubilizer, emulsifier, preservative and/or adjuvant. In certain embodiments, the therapeutic molecules can be formulated together or packaged together in a kit. In certain embodiments, the composition may be in a liquid or lyophilized form and comprises a diluent (Tris, acetate or phosphate buffers) having various pH values and ionic strengths, solubilizer such as Tween or Polysorbate, carriers such as human serum albumin or gelatin, preservatives such as thimerosal or benzyl alcohol, and antioxidants such as ascrobic acid or sodium metabisulfite. Also encompassed, in certain embodiments, are compositions comprising any of the therapeutic molecules modified with water-soluble polymers to increase solubility or stability. In certain embodiments, compositions may also comprise incorporation of any of the therapeutic molecules into liposomes, microemulsions, micelles or vesicles for controlled delivery over an extended period of time.

Specifically, in certain embodiments, compositions herein may comprise incorporation into polymer matrices such as hydrogels, silicones, polyethylenes, ethylene-vinyl acetate copolymers, or biodegradable polymers. Examples of hydrogels include, but are not limited to, polyhydroxyalkylmethacrylates (p-HEMA), polyacrylamide, polymethacrylamide, polyvinylpyrrolidone, polyvinyl alcohol and various polyelectrolyte complexes. Examples of biodegradable polymers include, but are not limited to, polylactic acid (PLA), polyglycolic acid (PGA), copolymers of PLA and PGA, polyamides and copolymers of polyamides and polyesters. Other controlled release formulations include, but are not limited to, microcapsules, microspheres, macromolecular complexes and polymeric beads which may be administered by injection.

Selection of a particular composition will depend upon a number of factors, including, but not limited to, the condition being treated, the route of administration, and the pharmacokinetic parameters desired. A more extensive survey of component suitable for pharmaceutical compositions is found in Remington's Pharmaceutical Sciences, 18th ed. A. R. Gennaro, ed. Mack, Easton, Pa. (1980), which is hereby incorporated by reference.

In certain embodiments, an effective amount or amounts of the therapeutic molecules will depend, for example, upon the therapeutic objectives, the route of administration, and the condition of the patient. Accordingly, in certain embodiments, the therapist may titer the dosage and modify the route of administration as needed to obtain the optimal therapeutic effect. A typical daily dosage may range from about 0.1 mg/kg to up to 100 mg/kg or more, depending on the factors mentioned above. In certain embodiments, a clinician can administer the composition or compositions until a dosage is reached that achieves the desired increase in erythropoiesis or clinical improvement of the blood disorder.

In certain embodiments, the composition or compositions may be administered as a single dose, or as two or more doses of one or more of the therapeutic molecules. These doses can consist of the same or different amounts of the therapeutic molecules and can be administered at the same or different times via the same or different routes of administration. In certain embodiments, the composition may be administered as a composition comprising any one or any combination of the therapeutic molecules. In certain embodiments, the combination may include the same or different amounts of the therapeutic molecules. In certain embodiments, the composition or compositions may be administered as a continuous infusion via implantation device or catheter. In embodiments in which the continuous infusion contains more than one therapeutic molecule, it may contain the same or different concentrations of the therapeutic molecules.

Determination of the optimum dosage, the optimum route of administration, and the optimum frequency of administration is well within the knowledge of those skilled in the art. For example, the agent of the present invention may be administered to the patient by one or more separate administrations, or by continuous infusion. For repeated administrations over several days or longer, depending on the condition, the treatment may be repeated until a desired suppression of disease symptoms occurs, a desired improvement in the patient's condition is achieved, or the desired level of EPO production is obtained. The dose may be readministered at intervals ranging from several times per week to once every six months.

Administration of the agent of the present invention can be combined with other therapies well-known to the skilled artisan for enhancing erythropoiesis, or for treating anemia, neurodegenerative diseases, inflammatory diseases, or acute ischemic or hemorrhagic stroke. In a preferred embodiment, the agent of the present invention is administered with one or more therapies for enhancing erythropoiesis. For example, in one embodiment of the present invention, a mammal may be administered a composition comprising the agent of the present invention, and an erythropoietin (EPO) receptor agonist. The erythropoietin (EPO) receptor agonist may be, but is not limited to, darbepoietin alfa (ARANESP™), epoietin alfa (EPO, EPOGEN™), and anti-EPO receptor agonist antibodies.

### EXAMPLES

### Example 1

### Generation of Monoclonal Antibodies that Specifically Bind Murine HVEM

To generate anti-mouse HVEM monoclonal antibodies, hamsters were immunized with a fusion protein in which the extracellular domain of murine HVEM was fused to the Fc region of murine IgG. For the first immunization, the fusion protein was emulsified with complete Freund's adjuvant (CFA). For the second and third immunizations, the fusion protein was emulsified with incomplete Freund's adjuvant (IFA). The immunizations were administered subcutaneously every 2 weeks. Following the third immunization, serum was harvested from the immunized hamsters, and the presence of anti-HVEM antibody was determined by ELISA. A fourth immunization was administered intraperitoneally in the absence of adjuvant. Five days after the fourth immunization, splenocytes were harvested and fused to Sp2/0 myeloma cells to generate monoclonal antibody-producing hybridomas. The culture supernatants of the resulting hybridomas were screened for the presence of antibodies which specifically bind murine HVEM by ELISA and flow cytometry.

### Example 2

### Identification of Antibodies Agonistic to HVEM

The anti-mouse HVEM antibodies generated in the experiments described in Example 1 were incubated with T-lymphocytes in 96 well plates coated with anti-CD3 antibody. Antibodies which stimulated the proliferation of the T-lymphocytes, as measured by an increase in incorporation of 3H-thymidine, were identified as being agonists of HVEM. One such antibody agonistic to HVEM was isolated and used for further experimentation, termed HM3.30.

### Example 3

### Characterization of the HM3.30 Mouse Monoclonal Antibody In Vivo

### Example 3.1

### Injection of HM3.30 Antibody Increases Spleen Erythroblasts

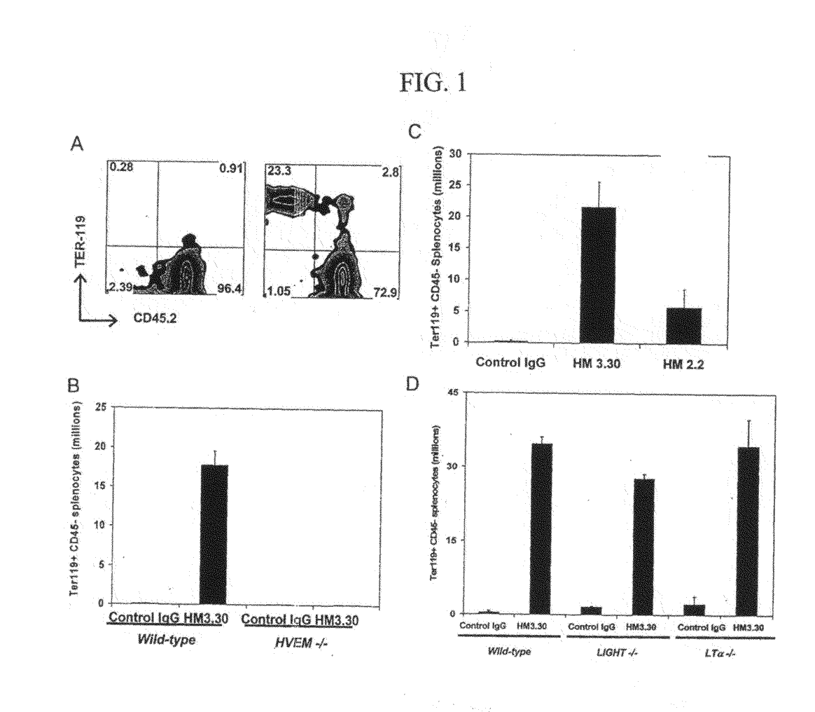

The function of the agonistic HM 3.30 antibody was assessed in vivo. Wild-type mice were injected intraperitoneally (i.p.) with either 150 μg of control IgG antibody (FIG. 1A, left box), or HM3.30 IgG antibody (FIG. 1A, right box). On day 4 after antibody administration, splenocytes from the mice were isolated and subject to fluorescence assisted cell sorting (FACS) using Ter-119 antibody and anti-CD45 antibody (BD Biosciences, CA). As shown in FIG. 1A, mice injected with the HM3.30 antibody exhibited an expansion of Ter-119⁺/CD45⁻ erythroid lineage cells by about 10-fold. The expansion of the Ter-119⁺/CD45⁻ erythroid lineage cells was determined to require HM3.30 antibody binding to HVEM, because the expansion of Ter-119⁺/CD45⁻ erythroid lineage cells in the spleen did not occur when the HM3.30 antibody was administered to HVEM knockout mice (HVEM^{-/-}) (n=3), or when a control antibody which does not bind HVEM was administered to wild-type mice (FIG. 1B) (n=2). Bars show the mean±SEM. In contrast to the agonistic anti-HVEM antibody HM3.30, a non-agonistic antibody, termed HM2.2, mediated considerably weaker expansion of Ter-119⁺/CD45⁻ erythroid lineage cells in the spleen (FIG. 1C). Bars show the mean±SEM of two mice per group.

The expansion of Ter-119⁺/CD45⁻ erythroid lineage cells induced by the HM3.30 antibody was not mediated by inhibition of binding of naturally-occurring ligands of HVEM. The HM3.30 antibody was administered to LIGHT^{-/-} mice (Tamada et al. (2000), J. Immunol., 164:4105), LTα^{-/-} mice (purchased from Jackson Laboratories), or wild-type mice. LIGHT^{-/-} mice and LTα^{-/-} mice demonstrated similar expansion of Ter-119⁺/CD45⁻ erythroid lineage cells in the spleen following administration of the HM3.30 antibody to the wild type control mice (FIG. 1D). Absolute numbers of Ter-119⁺/CD45⁻ splenocytes are shown with the mean±SEM. The expansion of the erythroid population was determined not to be a feedback response to erythrocyte lysis, as no evidence of anemia was found 24 hours after injection of the HM3.30 antibody.

### Example 3.2

### HM3.30-Mediated Erythropoietin Proliferation Originates in the Bone Marrow

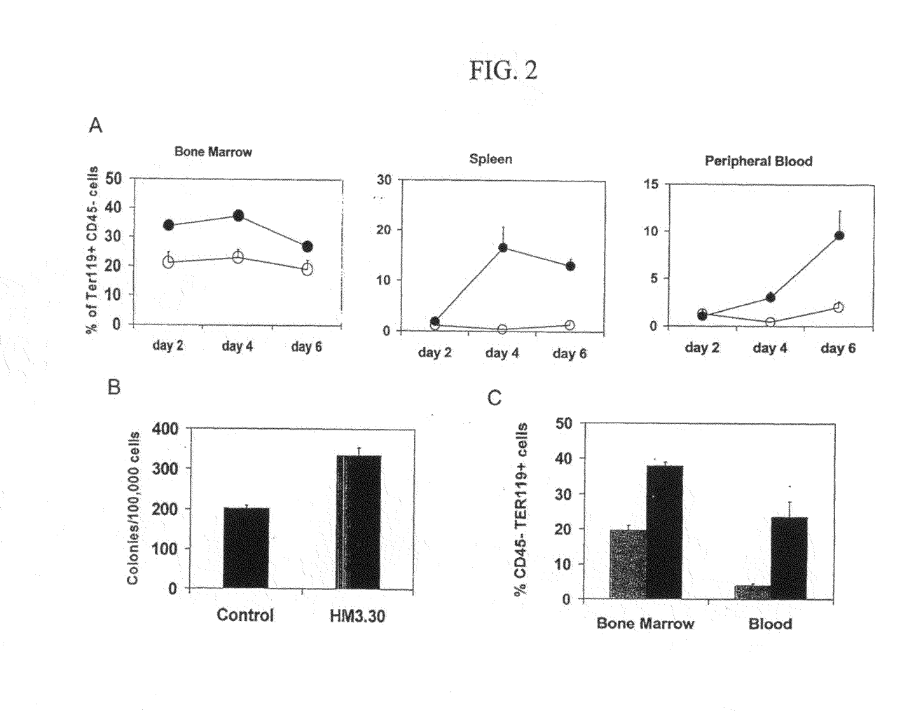

The origin of the Ter-119⁺/CD45⁻ erythroid lineage cells that undergo expansion upon administration of the HM 3.30 IgG antibody was determined by injecting 150 μg of HM3.30 IgG antibody, or 150 μg of control hamster IgG antibody, into wild-type mice. At days 2, 4, and 6 after antibody administration, the Ter-119⁺/CD45⁻ erythroid lineage cell populations were determined in the bone marrow, spleen and peripheral blood of the test mice (FIG. 2A). Each circle represents the mean±SEM of at least two mice. The expansion of the Ter-119⁺/CD45⁻ erythroid lineage cells in the bone marrow peaked at days 2 and 4, whereas the expansion of Ter-119⁺/CD45⁻ erythroid lineage cells in the spleen was not evident until day 4 after antibody administration. In the peripheral blood, the peak of Ter-119⁺/CD45⁻ erythroid lineage cell expansion did not occur until day 6 after antibody administration. The kinetics of the expansion in these different compartments indicated that the HM3.30 IgG antibody-induced expansion of Ter-119⁺/CD45⁻ erythroid lineage cells originated in the bone marrow.

In vitro plating of bone marrow cells on methylcellulose media, harvested from mice that had received the HM3.30 IgG antibody 12 hours prior, demonstrated significantly increased Benzidine positive, CFU-E numbers when compared to the bone marrow cells harvested from mice that had received the control hamster IgG antibody (FIG. 2B). Each bar represents the mean±SEM of 2 plates.

To determine whether the mechanism of HM3.30 antibody-induced erythroblast expansion resulted from downstream activation of HVEM signaling within the spleen, splenectomized mice, 2 days post-splenectomy, were injected with either the HM3.30 IgG antibody (black bars), or control hamster IgG antibody (grey bars). At 2 days after antibody administration, the expansion of Ter-119⁺/CD45⁻ erythroid lineage cells in the bone marrow and peripheral blood was measured (FIG. 2C). Bars show the mean±SEM of at least 3 mice. Erythrocyte expansion following administration of the HM3.30 IgG antibody occurred in the splenectomized mice, indicating that the spleen is not required for the Ter-119⁺/CD45⁻ erythroid lineage cell expansion.

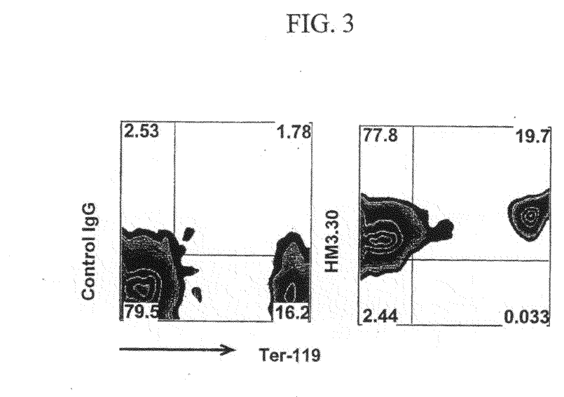

To determine whether the antibody could function directly on HVEM-expressing cells, Ter-119⁺ bone marrow cells were isolated and stained with anti-HVEM antibody. FACS analysis demonstrated that HVEM is ubiquitously expressed on Ter119⁺ cells, indicating that the antibody could have a direct effect on erythroid expansion in the bone marrow cells (FIG. 3).

### Example 4

### Induction of EPO Production by Administration of HM3.30 Antibody

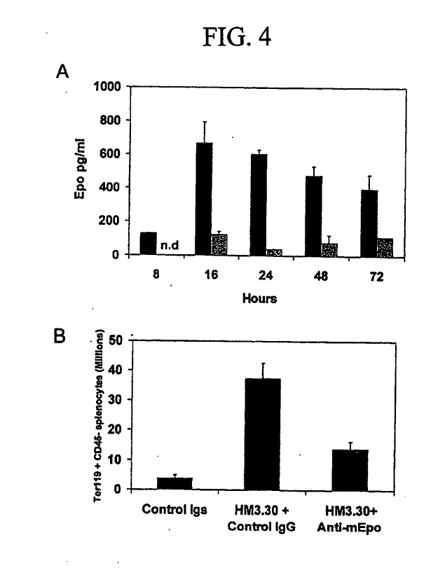

To determine the mechanism of expansion of the Ter-119⁺/CD45⁻ erythroid lineage cell population in the bone marrow, groups of wild-type mice were injected intraperitoneally with 150 μg of either control hamster IgG antibody, or with 150 μg of HM3.30 IgG antibody. At 8, 16, 24, 48 and 72 hours after antibody administration, mice were euthanized and the EPO levels in the serum were determined. In mice administered the HM3.30 IgG antibody, serum EPO levels were increased significantly, peaking at 24 hours after antibody administration (FIG. 4A). Each time point represents the mean±SEM of at least 4 mice. Pooled data from two independent experiments are shown. The expansion of the Ter-119⁺/CD45⁻ erythroid lineage cells was determined to be dependent upon EPO, because intraperitoneal administration of 150 μg of anti-EPO antibody, followed by intraperitoneal administration of 150 μg of HM3.30 IgG antibody, negated the expansion of Ter-119⁺/CD45⁻ erythroid lineage cells in the spleen (FIG. 4B). The absolute numbers of Ter-119⁺/CD45⁻ splenocytes on day 4 after antibody administration is shown in FIG. 4B. The bars represent the mean±SEM of 3 mice per group.

### Example 5

### Expression of HVEM on Bone Marrow Cells is Required for Expansion of Ter-119⁺/CD45⁻ Erythroid Lineage Cells, and EPO Production, upon Administration of HM3.30 Antibody

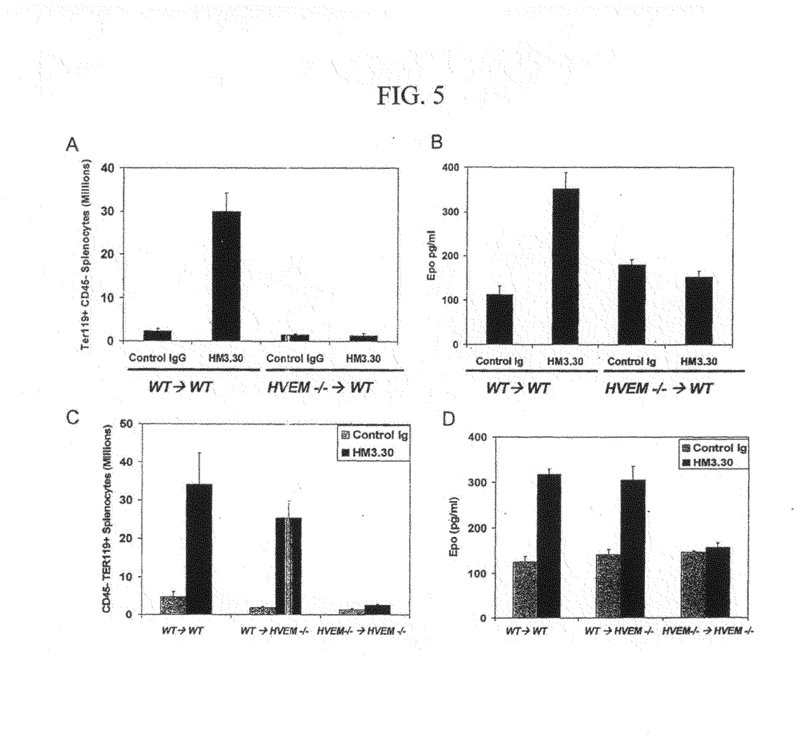

To determine whether agonistic anti-HVEM antibody can directly induce kidney interstital cells or epithelial cells to produce EPO, bone marrow chimeric mice were generated by transferring bone marrow cells from wild type or HVEM^{-/-} mice into irradiated wild type mice. Irradiated mice were prepared by irradiating the mice with 610 rads twice, with an interval of 6 hours between exposures. About five million donor bone marrow cells were transferred intravenously within 1 hour of the final irradiation treatment. Six weeks later, the chimeric mice were injected intraperitoneally with 150 μg of control IgG antibody or 150 μg of HM3.30 IgG antibody. The absence of HVEM on the hematopoietic cells ablated both the increase in EPO production (FIG. 5B) and the subsequent erythroid expansion (FIG. 5A), upon administration of HM3.30 IgG antibody. Conversely, in reverse chimeric mice, generated by transferring the bone marrow from wild type or HVEM^{-/-} mice into irradiated HVEM^{-/-} mice, the increase in EPO production (FIG. 5D) and the subsequent erythroid expansion (FIG. 5C) occurred upon administration of HM3.30 IgG antibody if the donor marrow was from wild type mice. These experiments indicate that expression of HVEM on bone marrow cells is required for expansion of the Ter-119⁺/CD45⁻ erythroid lineage cells, and the increase in EPO production, mediated by HM3.30 antibody. Each bar represents the mean±SEM of at least 4 mice.

### Example 6

### HM3.30 Antibody can Increase the Expansion of Ter-119⁺/CD45⁻ Erythroid Lineage Cells in the Spleen in the Absence of T-Lymphocytes, B-Lymphocytes, or Neutrophils

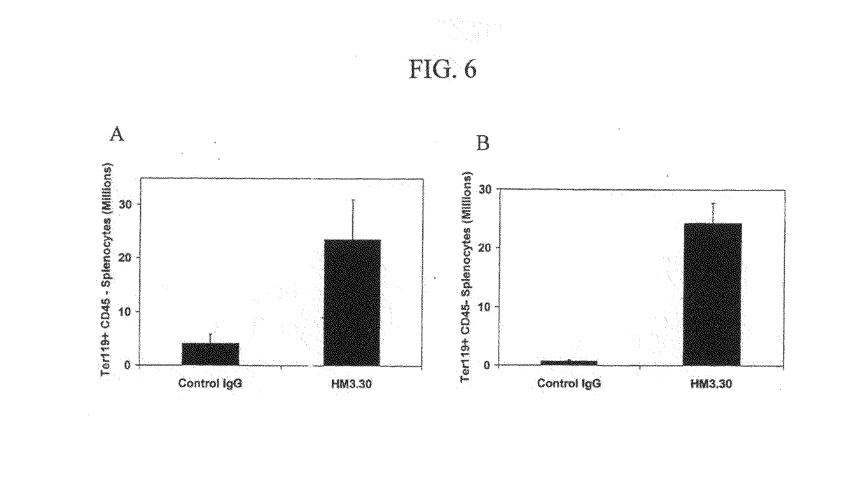

To determine whether agonistic anti-HVEM antibody can still induce erythroid expansion in the absence of T- and B-lymphocytes, Rag-deficient mice were injected intraperitoneally with either 150 μg of control Hamster IgG (n=2) or 150 μg of HM3.30 IgG antibody (n=3). At day 4 after antibody administration, the numbers of Ter-119⁺/CD45⁻ erythroid lineage splenocytes were determined. The absence of T- and B-lymphocytes had no observable effect on expansion of the Ter-119⁺/CD45⁻ erythroid lineage splenocytes mediated by HM3.30 (FIG. 6A). Furthermore, the HM3.30 antibody-mediated expansion of the Ter-119⁺/CD45⁻ erythroid lineage cells in the spleen was not negated by the depletion of neutrophils from Rag-deficient mice (FIG. 6B). In this experiment, neutrophils were depleted by administration of anti-Gr1.1 antibody at days −1 and 0 to the Rag-deficient mice. At day 0, the Rag-deficient mice were injected intraperitoneally with either 150 μg of control Hamster IgG (n=3) or 150 μg of HM3.30 IgG antibody (n=3). The experiments indicate that a population of bone marrow-derived cells, but which are not T-lymphocytes, B-lymphocytes, or neutrophils, mediate renal EPO production and erythroid expansion. The bars represents the mean±SEM.

### Example 7

### Inhibition of eNOS Reduces HM 3.30 Antibody-Mediated Erythropoiesis

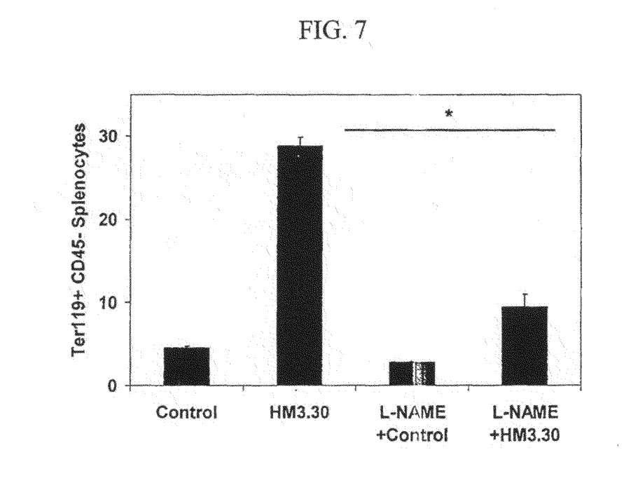

To further determine the mechanism of EPO production, inhibitors of downstream effectors of HVEM were analyzed for their effect on HM3.30-mediated expansion of Ter-119⁺/CD45⁻ erythroid lineage cells. Previous studies have shown prostaglandins and Nitric Oxide (NO) to be downstream effectors of HVEM (Chang et al., (2005), J. Biomed. Sci., 12:363; Heo et al., (2006), J. Leukoc. Biol., 79:330) Mice were administered 100 mg/kg of L-NAME, an inhibitor of eNOS (endogenous Nitric Oxide synthetase), intraperitoneally every 24 hours from day 0 to day 4. In addition, the mice were administered intraperitoneally either 150 μg of control Hamster IgG (n=3) or 150 μg of HM3.30 IgG antibody (n=3). Mice administered HM3.30 antibody and L-NAME exhibited about a 50% decrease in expansion of the Ter-119⁺/CD45⁻ erythroid lineage cell population (FIG. 7), indicating that NO may be a downstream mediator of the erythropoiesis induced by activation of HVEM. The bars represents the mean±SEM. “*” indicates that p<0.05 by student's t-test.

### Example 8

### Treatment of Cisplatin-Induced Anemia using HM3.30 Antibody

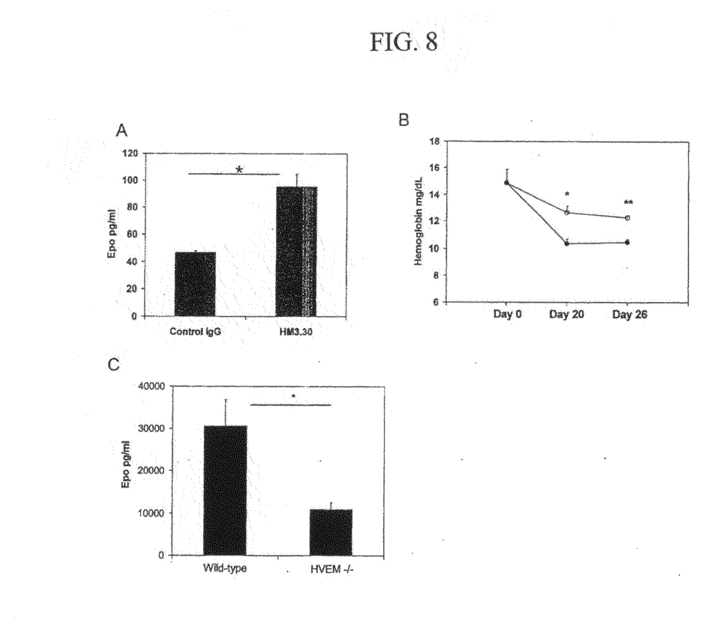

Cisplatin causes nephrotoxicity and has been shown to cause EPO-deficient anemia by killing the renal cells that produce EPO (Horiguchi et al:, (2006), Arch. Toxicol., 80:680). To determine whether administration of an anti-HVEM agonistic antibody can induce the production of EPO in an in vivo anemia disease model, mice were injected with 8 mg/kg of Cisplatin intravenously on day 0 and received either control IgG antibody (filled circles, FIG. 8A), or HM3.30 antibody (open circles, FIG. 8A) intraperitoneally on days 4, 8, 13, and 19. On day 20, the mice were euthanized, and serum EPO levels were determined. Serum EPO levels were increased in mice treated with HM3.30 IgG antibody over mice treated with control IgG antibody.

To induce overt anemia in mice, mice were administered 12 mg/kg of Cisplatin intravenously. The Cisplatin-treated mice were then administered either control IgG antibody (filled circles, FIG. 8B) or HM3.30 IgG antibody (open circles, FIG. 8B) on days 1 and 19. The mice were euthanized on days 0, 20 and 26, at which time blood hemoglobin levels were measured. Blood hemoglobin levels were significantly increased in Cisplatin-treated mice administered HM3.30 IgG antibody over Cisplatin-treated mice administered control IgG antibody. The circles represent the mean±SEM of at least 5 mice.

In addition, the role of HVEM in erythropoietic stress-induced EPO production was determined. Wild-type mice or HVEM^{-/-} mice were administered 150 mg/kg of 5-fluorouracil on day 0, and the levels of serum EPO were measured at day 9. HVEM^{-/-} mice exhibited a significantly decreased level of serum EPO in response to erythropoietic stress induced by 5-fluorouracil (FIG. 8C). The bars represent the mean±SEM of at least 4 mice. “*” indicates a student's t-test value of p<0.05.

### Example 9

### Binding Specificity of HM3.30 Antibody

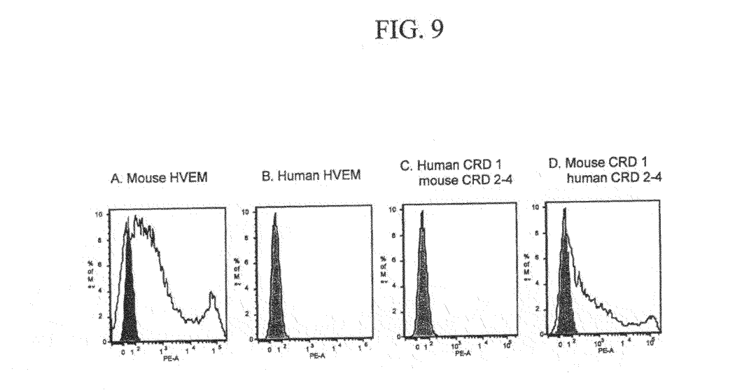

293T cells were transfected with plasmids encoding mouse HVEM (FIG. 9A), human HVEM (FIG. 9B), a chimeric gene of cysteine-rich domain (CRD) 1 of human HVEM fused to CRDs 2-4 of mouse HVEM (FIG. 9C), or a chimeric gene of CRD 1 of mouse HVEM fused to CRDs 2-4 of human HVEM (FIG. 9D). The transfected cells were labeled with the HM3.30 antibody, followed by PE-conjugated anti-hamster IgG. Antibody labeling was analyzed by flow cytometry. As a negative control, 293T cells were transfected with empty vector, and labeled with HM3.30 and PE-conjugated anti-hamster IgG (FIG. 9, filled histogram). HM3.30 specifically bound to CRD1 of mouse HVEM, but not to human HVEM. This indicates that HM3.30 antibody binds HVEM through an interaction with CRD 1. Without being bound by theory, an agonistic antibody of the invention has at least one requirement for stimulating erythropoiesis, which includes an interaction with CRD 1 (including, for example, binding thereto or otherwise interacting therewith).

### Example 10

### Generation of Agonistic Anti-Human HVEM Antibodies

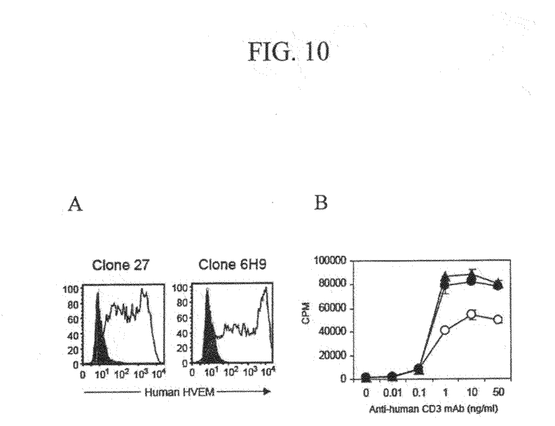

To generate anti-human HVEM monoclonal antibodies, BALB/c mice were immunized with a fusion protein in which the extracellular domain of human HVEM was fused to the Fc region of human IgG. For the first immunization, the fusion protein was emulsified with complete Freund's adjuvant (CFA). For the second and third immunizations, the fusion protein was emulsified with incomplete Freund's adjuvant (IFA). The immunizations were administered subcutaneously every 2 weeks. Following the third immunization, serum was harvested from the immunized hamsters, and the presence of anti-HVEM antibody was determined by ELISA. A fourth immunization was administered intraperitoneally in the absence of adjuvant. Five days after the fourth immunization, splenocytes were harvested and fused to Sp2/0 myeloma cells to generate monoclonal antibody-producing hybridomas. The culture supernatants of the resulting hybridomas were screened for the presence of antibodies which specifically bind murine HVEM by ELISA and flow cytometry. Two clones were identified that specifically bound to human HVEM (i.e., Clones 27 and 6H9), by measuring antibody binding to 293T cells expressing human HVEM. The intensity of labeling was detected by flow cytometric analysis (FIG. 10A). Antibody clones 27 and 6119 were also determined to be agonistic to human HVEM (FIG. 10B). Human T-cells were isolated from healthy donor PBMC and incubated in 96-well tissue culture plates pre-coated with antimouse IgG antibody, in the presence of the indicated doses of anti-CD3 monoclonal antibody and the anti-HVEM monoclonal antibodies (closed circles; clone 27, closed triangles; clone 6H9) or control mouse IgG (open circles). After 3 days of incubation, the proliferative activity of the T-cells was assessed by 3H-thymidine incorporation. The data points represent the means±the SD from triplicate wells. The data represent one of three independently repeated experiments.

### Example 11

### Agonistic Anti-Human HVEM Antibodies Stimulate Erythropoiesis In Vivo

The ability of agonistic anti-human HVEM antibodies (including, for example, antibodies derived from clones 27 and 6119 described herein) to stimulate erythropoiesis in vivo is evaluated using a preclinical mouse xenograft model. To this end, for example, NOD-scid/IL-2 receptor common gamma chain-deficient mice (available from Jackson Lab) are used. In these mice, an intravenous injection of a relatively low number (e.g., 5-20 million) of human peripheral blood mononuclear cells (PBMCs) result in an efficient engraftment of human hematopoietic cells for several weeks, a sufficient time frame for these experiments. Human PBMC-reconstituted mice are treated with an agonistic anti-human HVEM antibody, and the increase of serum Epo level and erythropoiesis in lymphoid organs are assessed by methods described herein and other methods known to one of ordinary skill in the art. In addition, an anemic condition is induced in the xenograft model by injecting cisplatin, followed by treatment with an agonistic anti-human HVEM antibody. Since HVEM expression on hematopoietic cells is necessary and sufficient for an erythropoietic effect of an agonistic anti-human HVEM antibody (as shown herein), this human PBMC reconstitution system is a valid preclinical model to screen and evaluate the activity of an agonistic anti-human HVEM antibody in vivo. Without being bound by theory, the administration of an agonistic anti-human HVEM antibody promotes erythropoiesis and is thus capable of treating anemia.

### REFERENCES

All patents and publications mentioned in this specification are indicative of the level of those skilled in the art to which the invention pertains. All patents and publications cited herein are incorporated by reference in its entirety to the same extent as if each individual patent or publication was specifically and individually indicated as having been incorporated by reference in its entirety.

1. An isolated antibody that specifically binds to a mammalian HVEM protein, wherein said antibody is an agonist of said mammalian HVEM protein, wherein said antibody induces erythropoiesis.

2. The isolated antibody of claim 1, wherein said HVEM protein is human HVEM protein.

3. (canceled)

4. The isolated antibody of claim 1, wherein said antibody is an IgG antibody.

5. The isolated antibody of claim 1, wherein said antibody is selected from the group consisting of a monoclonal antibody, a polyclonal antibody, a humanized antibody, and an antigen-binding antibody fragment thereof.

6. The isolated antibody of claim 1, wherein said antibody binds cysteine-rich domain 1 (CRD-1) of HVEM protein.

7. The isolated antibody of claim 1, wherein the light chain variable region of said antibody comprises the amino acid sequence of SEQ ID NO: 1.

8. The isolated antibody of claim 1, wherein the heavy chain variable region of said antibody comprises the amino acid sequence of SEQ ID NO: 2.

9-11. (canceled)

12. An isolated antibody that specifically binds to the polypeptide of SEQ ID NO: 3, wherein said antibody stimulates erythropoiesis.

13-14. (canceled)

15. A method for stimulating erythropoiesis in a mammal, comprising administering to said mammal an effective amount of a composition comprising an antibody that activates herpes virus entry mediator (HVEM) protein of said mammal, thereby stimulating erythropoiesis in said mammal.

16. The method of claim 15, wherein said antibody is an agonist of HVEM protein.

17. The method of claim 16, wherein said antibody is selected from the group consisting of a monoclonal antibody, a polyclonal antibody, a humanized antibody, an antigen-binding antibody fragment thereof, and a single-chain antibody.

18. The method of claim 17, wherein said antibody is a monoclonal antibody.

19. The method of claim 18, wherein said antibody is an IgG antibody.

20. (canceled)

21. The method of claim 15, wherein said antibody binds cysteine-rich domain 1 (CRD-1) of HVEM protein.

22-24. (canceled)

25. The method of claim 15, wherein said composition is administered intravenously, intravascularly, subcutaneously or intraperitoneally.

26. The method of claim 15, wherein said mammal has an anemic disorder.

27. (canceled)

28. The method of claim 15, wherein said mammal is a human.

29. The method of claim 15, wherein said administering of a composition comprising an agent which activates HVEM protein further comprises administering an erythropoietin (EPO) receptor agonist.

30. (canceled)

31. The isolated antibody of claim 12, wherein the antibody stimulates production of erythropoietin in the mammal.

32. The method of claim 15, wherein said antibody stimulates production of erythropoietin in the mammal.


SEQUENCE LISTING (3 sequences)

SEQ ID NO: 1 - DNA, 399 bp, Cricetulus migratorius
  HM3.30 antibody light chain variable region
atggaatcacatatccatgtcttcatgttcttgttcctttgggtgtctggttcctgtgcagacatcgtgatgacccagtctccatcatccctcgctgtgtcagcaggagagaaggccactatcagctgccagtccagtcagagtcttatcaacagttacaccaataagaactatttgaactggtacctgcagagaccagggcagtctcctaagctgctgatctaccatgcatccactaggcatactggggtccctgatcactacataggcagtggatctgggactgatttcactctcaccatcagcagtgtccaggctgaagacctgggagactattactgtcagcagcattatggatctccgtggacgttcggacctggcaccaagctggagatcaaa

SEQ ID NO: 2 - DNA, 363 bp, Cricetulus migratorius
  HM3.30 antibody heavy chain variable region
gagatccaattgcaacaatttgggcctcagctggtaaggcctggttcctcagtcaaggtgtcctgccaggtttcaggctacaacatcattgagtattatgtacattgggtgaagcagaagactggacagggactggagtggatgggacgtctcaaccccagaagtggtagcacagactatgcacagaagttccatggacgggtctccatgaccagtgacacatcttccagcacagcctacatggaactcagcagcctgacatctgaggactctgcagtctatttctgtacaaatagcggatacagaagcctctatggttatgttgatgtctggggccaggggatccaggtcaccgtctcctca

SEQ ID NO: 3 - protein, 283 aa, Homo sapiens [sequence omitted]